(19) United States
(12) Patent Application Publication
(10) Pub. No.: US 2004/0148645 A1
(43) Pub. Date: Jul. 29, 2004

(54) ATP diphosphohydrolase (CD39) gene therapy for inflammatory or thrombotic conditions and transplantation and means therefor

(76) Inventors: Fritz H. Bach, Boston, MA (US); Simon Robson, Brookline, MA (US); Adrien R. Beaudoin, Rock Forest (CA); Jean Sevigny, Brookline, MA (US)

Correspondence Address: NOVARTIS, CORPORATE INTELLECTUAL PROPERTY, ONE HEALTH PLAZA 430/2, EAST HANOVER, NJ, 07936-1080, US

(21) Appl. No.: 10/756,572

(22) Filed: Jan. 13, 2004

Related U.S. Application Data
(63) Continuation of application No. 09/234,286, filed on Jan. 20, 1999, now abandoned.
Application No. 09/234,286 is a continuation of application No. 08/600,383, filed on Feb. 12, 1996, now abandoned.
Application No. 08/600,383 is a continuation-in-part of application No. 08/410,371, filed on Mar. 24, 1995, now abandoned.

(51) Int. Cl. A61K 48/00; A01K 67/027

(52) U.S. Cl. 800/8; 424/93.21

(57) ABSTRACT

A method to render endothelial cells capable of inhibiting platelet and leukocyte-mediated injury and inflammation is described, comprising genetically modifying the cells by inserting DNA encoding ecto-ATP diphosphohydrolase or an oxidation-resistant analog thereof, and expressing a protein having functional ecto-ATP diphosphohydrolase activity, such as the human CD39 protein, by said cells under cellular activating conditions. The method, which can be carried out in vivo, ex vivo or in vitro, has use in allogeneic or xenogeneic transplantation as well as to treat systemic or local inflammatory conditions characterized by platelet aggregation leading to thrombus formation.

This application is a continuation-in-part of application Ser. No. 08/410,371 filed Mar. 24, 1995.

### FIELD OF THE INVENTION

The present invention provides improvements in the field of gene therapy and tissue and organ transplantation.

The invention in its broad aspect is concerned with genetic modification of endothelial cells to render such cells less suceptible to an inflammatory or other activating stimulus.

In particular, the invention is addressed to genetic modification of endothelial cells subject to a platelet-mediated activation stimulus, to render them capable of inhibiting platelet aggregation by expressing functional ATP diphosphohydrolase activity under conditions of endothelial cell activation and inflammation.

In a preferred embodiment, the invention is addressed to a novel use of the polypeptide or class of polypeptides previously identified as a B cell activation marker, CD39. It has now been found that the aforementioned CD39, a cell surface glycoprotein associated with B lymphocytes, activated NK cells, certain T cell and endothelial cells, but heretofore unassigned a cell-specific function, exerts an ATP- and ADP-degrading, i.e. ATP-diphosphohydrolase, activity. The novel use of said CD39 which is contemplated by this, invention therefore comprises the suppression or inhibition of ADP-induced platelet aggregation and thrombus formation, particularly under cellular activating conditions or in connection with tissue inflammation. Accordingly, the invention in its further aspects and embodiments is concerned with genetic modification of mammalian cells, and tissues or organs comprising said cells, to render such cells, organs or tissues capable of expressing CD39 protein, and maintaining the function of expressed protein at sufficient levels under cellular activating conditions, whereby platelet aggregation at the surface of said cells (and, ultimately, thrombus formation) are suppressed or inhibited.

The invention also contemplates use of CD39 protein (gene) in connection with such further embodiments as are disclosed herein in general for an ATP diphosphohydrolase active protein.

The invention is also concerned with methods of transplantation of genetically modified cells, or graftable tissue or organs comprising said cells; and most particularly is directed to methods of transplanting modified xenogeneic or allogeneic cells, tissues or organs, recombinant vectors for accomplishing same, and the cells, tissues or organs, as well as non-human transgenic or somatic recombinant animals, so modified.

The invention also provides oxidation resistant analogs of the involved ATP diphosphohydrolase (e.g., CD39) protein.

The invention further concerns a method of inhibiting platelet aggregation in a mammal comprising administering to said mammal an effective amount for inhibiting platelet aggregation of a polypeptide having ATP diphosphohydrolase activity, or pharmaceutically acceptable salt thereof, in a pharmaceutically acceptable carrier; and compositions therefor.

### BACKGROUND OF THE INVENTION

Thromboembolic phenomena are involved in a number of vascular diseases and pathologies, including a variety of atherosclerotic and thrombotic conditions, for example, acute myocardial infarction, chronic unstable angina, transient cerebral ischemic attacks and strokes, carotid endarterectomy, peripheral vascular disease, restenosis, and/or thrombosis following angioplasty, or anastomosis of cardiovascular devices, such as catheters or shunts. Also relevant are preeclampsia, as well as vasculitis (e.g., Takayasa's disease, and rheumatoid vasculitis).

In the field of allogeneic or xenogeneic transplantation, as well, thrombus formation in the vasculature of grafts is a serious problem affecting the viability of implanted tissues and organs.

A recognized component of the body's complex physiological mechanism for forming a thrombus is the sequence of events giving rise to platelet activation (also referred to as platelet “adhesion” and “aggregation”).

In brief, the endothelium (also known as the “vascular endothelium”) consists of a layer of cells that line the cavities of the heart and of the blood and lymph vessels. The process of “activation” of endothelial cells by platelet and leukocyte mediated injury and inflammation, with accompanying release of activating agents, such as the cytokine, TNFα, has been described in the literature, see Bach et al., Immunological Reviews (1994) 141. 5-30; Pober and Cotran, Transplantation (1991) 52 1037-1042 (both incorporated by reference). A phenomenon associated with this process is the retraction of the endothelial surface and exposure of constituents of the subendothelial matrix, such as collagen and von Willebrand Factor (vWF).

Concomitant with endothelial “activation,” the platelets, normally freely circulating in the blood, also become “activated” by the exposed constituents of the subendothelial matrix, as well as by thrombin and activated complement components. In this activated state, enhanced expression of platelet glycoprotein (GP)IIb/IIIa and P-selectin promotes affinity for components of the endothelium and subendothelium. Additionally, platelets begin to secrete biologically active constituents, in particular, the adenine nucleotides, ATP and ADP. ADP is essential for continued platelet activation responses and leads to further recruitment of platelets. ATP also stimulates neutrophils via their P2y receptors and results in the increased release of reactive oxygen intermediates. In a continuing inter-related sequence of events, platelet “aggregation” is initiated by the binding of agonists such as ADP, as well as thrombin, epinephrine, ADP, collagen and thromboxane A2, to platelet membrane receptors. Stimulation by agonists results in exposure of latent fibrinogen receptors on the platelet surface, and finally, the binding of fibrinogen to the platelet GPIIb/IIIa receptor complex, which is believed to be principally responsible for platelet aggregation and thrombus formation in vivo.

Opposing the above-described platelet aggregation process are various potent antithrombotic and fibrinolytic mechanisms, which are primarily localized to the endothelium, e.g., (i) release of prostacyclines, (ii) generation of nitric oxide and (iii) activity of ADP-degrading enzymes. However, it is self-evident that these mechanisms may be ineffective and are unable to prevent many inflammatory vascular disorders, or to maintain graft survival, with the result that platelet activation and aggregation proceed, largely unregulated, to ultimate vascular occlusion and platelet thrombosis.

Graft injury and loss seen with graft preservation-induced endothelial damage, as well as in allograft and xenograft rejection, exemplify the vulnerability of endothelial tissue in the activated condition to thrombotic complications.

For example, following anastomosis of the vasculature of a graft, recipient platelets begin to interact with endothelial and subendothelial cells of the graft. Activation of the graft endothelium in an inflammatory environment can initiate the platelet aggegation cascade, with consequent adhesion and aggregation of the platelets on the graft endothelium, rendering the graft susceptible to thrombosis and, ultimately, graft failure.

Considerable effort by workers in the art has been directed toward elucidation of agents which can control platelet aggregation. However, antiplatelet agents currently in clinical use have recognized side-effects, and suffer lack of selectivity. Newer GPIIb/IIIa antagonists, such as peptides, peptidomimetics and antibodies, are more selective and potent but do not serve a prophylactic function in the early stages of inflammation or injury. Certain purinergic P2T receptor antagonists, and to some extent PAF antagonists, have similar shortcomings.

There exists a critical need for a method to prevent or minimize platelet aggregation occurring in connection with endothelial cell activation. In particular, there is a need to prolong graft organ survival, while minimizing toxicity and other adverse effects associated with available platelet activation inhibitors.

### SUMMARY OF THE INVENTION

The inventors have discovered that regulation and inhibition of platelet aggregation under cellular activating conditions are critically dependent on the maintenance of an ecto ATP-diphosphohydrolase activity by endothelial cells.

More particularly, it has been discovered by the inventors that activation of endothelial cells (hereinafter “EC”) in response to an immune or inflammatory stimulus leads to the reduction or loss of the ADP-hydrolyzing activity on the surface of said cells; and furthermore, this reduction or loss of ADP-hydrolyzing activity results in platelet adhesion to the endothelial cell surface and platelet aggregation, and ultimately leads to thrombus formation.

For example, the inventors have observed that EC, in the absence of activating agents, can express a cell-associated ATP-diphosphohydrolase activity which is capable of inhibiting platelet activation. The inventors have found that under conditions promoting activation of said EC (e.g., exposure to TNFα/complement and hyperacute rejection of a xenograft/reperfusion injury/oxidative stress), there is a reduction or loss of said ecto ATP-diphosphohydrolase activity, resulting in a cellular environment with increased susceptibility to platelet aggregation.

The inventors have further discovered that the activity of native mammalian/porcine ATP diphosphohydrolases is suceptible to oxidation, and when oxidized, the protein loses the ability to suppress platelet activation. It is now believed that this phenomenon plays a significant role in many pathogenic states, including platelet aggregation and thrombus formation seen with graft rejection.

It is noted that many of the pathologies or disease conditions requiring therapy directed toward suppressing platelet aggregation are associated with high levels of toxic oxygen radicals and other reactive oxygen intermediates. An example of such a pathology is graft preservation injury and ischemia-reperfusion. Implicated disease states are reperfusion injury associated with myocardial infarction, disseminated intravascular coagulation associated with septicemia, alveolar fibrosis associated with adult respiratory syndrome, and noncardiogenic pulmonary edema. Furthermore, injury to the endothelium involves the influx of activated monocytes, polymorphonuclear leukocytes, etc., which can also create toxic oxygen species.

While workers in the art hitherto recognized a general connection between endothelial cell damage, inflammation and thrombosis, the inventors are the first to recognize that the enzyme, ATP diphosphohydrolase, under conditions of oxidant stress, exhibits diminished ability to prevent platelet aggregation. This is a novel observation critically important in the treatment of many of the pathological conditions requiring restoration of a cellular platelet activation-suppressing, or anti-thrombotic function.

We have also found that significant (e.g. 95% or greater, typically 98% or greater, e.g., 99% and greater, and even 100%) homology exists between peptide sequences corresponding to type I and type II ecto-ATP diphosphohydrolases, such as reported by Christoforidis and co-workers (Eur. J. Biochem. 234(1):66-74, 1995 Nov. 15, which is hereby incorporated by reference), and the CD39 lymphocyte activation marker (accession 765256; 23 Mar. 1995) cloned from a human B cell lymphoblastoid cell line by Maliszewski and co-workers, J. Immunol., 1994, 153:3574-3583, incorporated by reference.

Accordingly, it is our observation, previously unappreciated in the art, that the CD39 protein or class of proteins encodes an ATP hydrolyzing function, and in particular, an ecto-ATP diphosphohydrolase.

Therefore, the term “ATP diphosphohydrolase” or “ecto-ATP diphosphohydrolase” employed herein shall be understood to refer to and include native CD39 protein (especially, native human CD39 protein).

Accordingly, the invention in its broader aspects concerns a method of genetically modifying endothelial cells to render them less susceptible to an inflammatory or immunological stimulus and platelet adhesion by conferring on said cells the capability of “stably” expressing ATP diphosphohydrolase activity under cellular activating conditions, i.e. expressing ATP diphosphohydrolase at sufficient levels whereby platelet adhesion or aggregation at the cell surface are suppressed or inhibited.

Thus by “stable” expression is meant that transcription and expression of the ATP diphosphohydrolase protein (or analog) by the cell is maintained at antithrombotic (i.e. platelet plug/thrombosis-suppressing) effective amounts. Such concentrations of the protein may be the same, higher or even lower than is expressed by the cell under hemostatic conditions; however, such “stable” expression of the ATP diphosphohydrolase protein is sufficient to result in a reduction or suppression of platelet aggregation and platelet thrombi in the vasculature in the local micro-environment of the cell, i.e. at the surface of the modified cell, as compared to a cell under similar activation conditions which is not modified according to the invention (i.e. does not contain the inserted gene/protein).

By “cellular activation conditions” is meant Type I EC activation (referring to early events following stimulation, which include the retraction of EC from one another as well as hemorrhage and edema); and/or Type II EC activation (referring to later events which occur over hours and are dependent upon tanscriptional regulation and protein synthesis) (see Bach et al., id.).

A generally accepted indicator of Type I EC activation is an elevated level of PAF and/or P-selectin in the cellular environment.

A generally accepted indicator of Type II EC activation is an elevated level of E-selectin in the cellular environment or membranes.

Suppression or inhibition of platelet aggregation at the surface of a cell modified according to the invention can be determined by known methods. A reduction in platelet aggregate formation at the surface of the cell of 50% and greater, and preferably 65% and greater, demonstrates platelet inhibition or suppression for purposes of the invention.

The stable, or high-level, ADP-hydrolyzing activity provided by the invention can be obtained using vector constructs encoding the ATP diphosphohydrolase protein under the control of a promoter that will be functional (active) even under conditions of EC activation or oxidative stress, and thus replace the activity of the normally present ATP diphosphohydrolase. Examples of such promoters include “constitutive” or “inducible” promoters.

By “constitutive” is meant that protein expression is essentially independent of cellular activation factors, and is essentially continuous over the life of the cell.

By “inducible” is meant that protein expression can be controlled by administration of exogenous factors either not typically present in the cellular environment, or lost or diminished from the cellular environment under activating conditions. Such exogenous factors may include cytokines or growth factors.

It is also within the contemplation of this invention to achieve “stable” ATP-diphosphohydrolase activity by providing peptides that have ADP-hydrolyzing activity under oxidizing conditions. Thus the invention provides peptide analogs having activity of a native ATP-diphosphohydrolase (e.g., CD39) which are substantially oxidation-resistant.

Also contemplated is co-administration of an anti-oxidant to the affected cell, tissue or organ, concomitant with expression of the ecto-ATP diphosphohydrolase.

Accordingly, the invention in its more particular aspects comprises a method of modifying endothelial or other mammalian cells (e.g., monocytes, NK cells, lymphocytes, islet cells) by inserting into such cells, or the progenitors of said cells, DNA encoding functional ecto-ATP diphosphohydrolase protein or an oxidation-resistant analog thereof in operative association with a promoter, and stably expressing ecto-ATP diphosphohydrolase from said cells under cellular activating conditions, i.e. whereby platelet aggregation at the surface of the cell is reduced or suppressed.

By “functional” is meant that the expressed ATP-diphosphohydrolase of said cell hydrolyzes platelet secreted ADP to AMP and monophosphate.

The invention also comprises a method of controlling platelet aggregation, and thereby preventing or alleviating a thrombotic condition, in a mammalian subject in need of such therapy, comprising genetically modifying endothelial cells of said patient by inserting therein DNA encoding ATP diphosphohydrolase, or an oxidation-resistant analog thereof, in operative association with a suitable promoter, and expressing functional ecto-ATP diphosphohydrolase from said cells at thrombus-suppressing effective levels under cellular activating or inflammatory conditions, whereby platelet aggregation is suppressed.

Preferably the cells or tissue are modified in vivo, i.e. while remaining in the body of the subject.

In another aspect, cell populations can be removed from the patient, genetically modified ex vivo by insertion of vector DNA, and then re-implanted into the subject.

The subject is preferably human.

In a further aspect, the invention comprises a method of transplanting donor allogeneic or xenogeneic endothelial cells, or graftable tissue or organs comprising said cells, to a mammalian recipient in whose blood or plasma said cells or tissue are subject to activation, which comprises:

(a) genetically modifying said cells, or the progenitor cells thereof, by inserting therein DNA encoding ATP-diphosphohydrolase protein or an oxidation-resistant analog thereof in operative association with a promoter; and

(b) transplanting said modified donor cells, tissues or organs into said recipient and expressing from the so-modified cells or tissue functional ADP-hydrolyzing enzyme under cellular activating conditions, i.e. whereby platelet aggregation at the cellular surface is reduced or suppressed.

(The “modified donor cells” of step (b) will be understood to refer to cells which themselves were subject to genetic modification in step (a) as well as to progeny thereof.)

Steps (a) and (b) may be carried out in either order; that is, the donor allogeneic or xenogeneic cells, tissue or organs, may be modified or genetically engineered (e.g., by transfection, transduction, transformation or the like) prior to, or alternatively after, implantation into the recipient.

For example, endothelial cells from tissue or organs of a pig may be genetically modified in vivo by insertion of DNA encoding human ATP-diphosphohydrolase protein or oxidation-resistant analog under the control of a promoter, and the modified cells or tissue are then recruited for grafting into a human recipient. Once transplanted, the transgenic cells or tissue or organs express functional human ecto-ATP-diphosphohydrolase or an oxidation-resistant analog, even in the presence of otherwise down-regulatory factors and in an inflammatory environment.

Since porcine or bovine ATP-diphosphohydrolase factors, for example, have cross-species activity, porcine or bovine protein-expressing transgenic (or somatic recombinant) animals may usefully be employed for recruitment of cells, tissues and organs for transplantation to humans. Preferably, however, the human protein or analog in a suitable vector will be used to modify porcine donor cells or organs to render them transgenic (or somatic recombinant) for transplantation purposes.

Somatic recombinant or transgenic donor animals can be obtained by modifying cells of the animal, or earlier, e.g., at the embryonic stage, by well-known techniques, so as to produce an animal expressing the desired protein.

Donor cells or tissue can also be genetically modified ex vivo, whereby cells, tissues or organs extracted from the donor and maintained in culture are genetically modified as above-described, and then transplanted to the recipient, where the graft can then express the desired functional protein.

It is preferable that the genetic modification of the donor be done in vivo.

According to a further aspect of the invention, there are provided graftable endothelial cells, tissue or organs of a donor species, the cells, tissue or organ being modified to stably express functional ATP-diphosphohydrolase in a graft recipient of the same or different species as the donor under cellular activating conditions.

In its additional aspects, the invention provides a non-human transgenic (or somatic recombinant) mammal having endothelial cells or tissue so-modified; and a method of preparing said non-human transgenic mammal. Such non-human transgenic animals are particularly of the procine species (although murine transgenics expressing human ATP diphosphohydrolase are also contemplated to be within the scope).

Also disclosed is a means of treating thrombotic disorders in a mammalian (i.e. human) subject, comprising administering to the subject a platelet thrombus-suppressing effective amount of ecto-ATP diphosphohydrolase protein or oxidation-resistant analog, and pharmaceutical compositions comprising said protein in soluble form.

Also contemplated is the coating of prosthetic intravascular devices with the recombinant produced protein or analog.

It will be apparent that such therapies will be useful to alleviate thrombotic conditions in a patient, and in particular to moderate thrombotic complications occurring in connection with organ transplantation, especially where the graft recipient is human.

### BRIEF DESCRIPTION OF DRAWINGS

FIG. 1: Bar graph depicting the inhibitory effect of human TNFα on ecto-ATP diphosphohydrolase activity.

FIG. 2: Reciprocal plot depicting the kinetics of quiescent cytokine mediated PAEC.

FIG. 3: Bar graph depicting peroxide and cytokine mediated loss of ecto-ATP diphosphohydrolase activity on PAEC.

FIG. 4: Bar graph demonstrating that mercaptoethanol (BME protects against cytokine mediated loss of ecto-ATP diphosphohydrolase activity on PAEC.

FIG. 5: Bar graph showing kinetics of ecto-ATP diphosphohydrolase modulation by TNFα and oxidants.

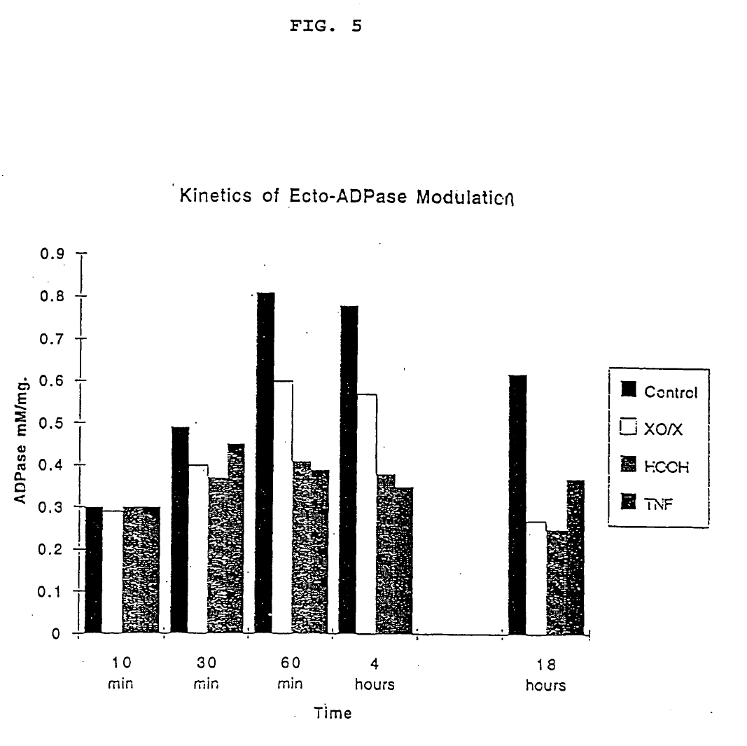

FIG. 6: Plot of ecto-ATP diphosphohydrolase activity of activated PAEC treated with antioxidants.

FIG. 7: Bar graph showing ecto-ATP diphosphohydrolase activity in purified rat glomeruli as a function of reperfusion time in vivo.

FIG. 8: Bar graph demonstrating effect of pre-treatment with cobra venom factor (CVF) of rat glomeruli rendered ischaemic and then reperfused.

FIG. 9: Northern analysis of CD39 in HUVEC following TNFα stimulation.

### DEFINITION OF TERMS

“Graft,” “transplant” or “implant” are used interchangeably to refer to biological material derived from a donor for transplantation in to a recipient, and to the act of placing such biological material in the recipient.

“Host or “recipient” refers to the body of the patient in whom donor biological material is grafted.

“Allogeneic” refers to the donor and recipient being of the same species (see also allograft). As a subset thereof, “syngeneic” refers to the condition wherein donor and recipient are genetically identical. “Autologous” refers to donor and recipient being the same individual. “Xenogeneic” (and “xenograft”) refer to the condition where the graft donor and recipient are of different species.

“ATP diphosphohydrolase”: an enzyme capable of catalyzing the sequentual hydrolysis of adenosine triphosphate (ATP) to adenosine diphosphate (ADP) to adenosine monophosphate (AMP) (the enzyme is also alternately referred to as ADPase; ATPDase; ATPase; ADP monophosphatase, or apyrase, EC 3.6.1.5)

The term “a polypeptide having activity of an ATP diphosphohydrolase” shall be understood to include native ecto-ATP diphosphohydrolase protein, as well as oxidation resistant peptide analogs thereof, and soluble truncated forms.

An example of an ecto-ATP diphosphohydrolase is the CD39 protein.

“CD39” refers to a natural mammalian gene (including cDNA thereof) or protein, including derivatives thereof having variations in DNA or amino acid sequence (such as silent mutations or deletions of up to, e.g., 5 amino acids) which do not prejudice the ATP-hydrolyzing activity of the protein. The CD39 gene (protein) employed in the invention may, for example, be porcine, bovine or human, or may of a primate other than human, depending on the nature of the cells to be modified and, for example, the intended recipient species for transplantation.

The term “human CD39” as used herein shall refer to a protein which is at least 70%, preferably at least 80%, more preferably at least 90% (e.g., 95% or greater, e.g. 99% or 100%) homologous to the amino acid sequence of the CD39 lymphocyte activation marker reported by Maliszewski and co-worker (Genbank/NCBI accession 765256; 23 Mar. 1995) in J. Immunol., 1994, 153(8):3574-83, which is incorporated herein by reference.

### DETAILED DESCRIPTION OF THE INVENTION

The ATP diphosphohydrolases comprise a family of proteins which catalyze the sequential phosphorolysis (i.e. removal of phosphate groups) of ATP to ADP to AMP. In general, proteins of this class exhibit nonspecificity toward nucleoside di- or triphosphates; and are activated by Ca²⁺ or Mg²⁺. By converting ADP into AMP, as well as ATP, via ADP, into AMP, these enzymes inhibit or reverse platelet aggregation. The final product, AMP, is a substrate for 5′ nucleotidases and generates adenosine, an important platelet anti-activator and vasodilator.

The proteins are primarily found in the cellular elements of the blood and the vascular wall. For such cellular enzymes to be effective, the enzymes should be functional at the cell surface, i.e. as ecto-enzymes. Because the ATP diphosphohydrolases are membrane-associated, insoluble proteins expressed on the cell surface, they are conventionally referred to as ecto-ATP diphosphohydrolases. Soluble analogs of said protein may also be prepared by known methods to be infused. For example, soluble analogs can be obtained by treating the full length protein with standard detergents. Alternatively, a DNA construct can be prepared which contains the DNA encoding the functional protein, from which the membrane-spanning sequence of the gene is deleted, thereby rendering the expressed protein soluble and/or secretable through the endothelial cell membrane into the immediate environment within the vasculature.

The activity of ecto-ATP-diphosphohydrolases has been demonstrated on endothelial cells, as well as leukocytes and platelets, and these proteins are believed to be widely distributed over the mammalian vascular endothelium.

Christoforidis and co-workers, 1995, id. disclosed partial internal amino acid sequence information following chymotryptic cleavage of an ATP diphosphohydrolase isolated from the particulate fraction of human term placenta.

Purification of bovine aortic and iliac endothelial ecto-ATPase was reported in a presentation and abstract by Sevigny and co-workers (University of Sherbrooke, Canada) at the IBC Anticoagulant and Antithrombotic meeting in Boston, Oct. 24-25, 1994 (the abstract of which is incorporated by reference).

Additionally, Lin and Guidotti, J. Biol. Chem., Vol. 264, No. 24, 14408-14414 (1989), reported possession of rat liver CAM-105 cDNA and polyclonal antibodies, as well as identifying a consensus sequence (GPAYSGRET amino acids 92-100) within the protein, and prepared oligonucleotide primers corresponding to nucleotides −40 to −24 (5′) and 473 to 496 (3′); see also Sippel et al., J. Biol. Chem., 264:4, 2800-2826 (1994); Cheung et al., J. Biol. Chem., Vol. 268, No. 32, 24303-24310 (1993). Further work has been reported in connection with the characterization of an ATP diphosphohydrolase active in rat blood platelets, Frasetto et al. Molecular and Cellular Biochemistry 129:47-55, 1993; the characterization of ATP-diphosphohydrolase activities in the intima and media of the bovine aorta, Cote et al., Biochimica et Biophysica Acta, 1139 (1992) 133-142; the purification of ATP diphosphohydrolase from bovine aorta microsomes, Yagi et al., Eur. J. Biochem. 180, 509-513(1989); and the characterization and purification of a calcium-sensitive ATP diphosphohydrolase from pig pancreas, LeBel et al., J. Biol. Chem., Vol. 255, No. 3, 1227-1233 (1980); all of the abovementioned publications being incorporated herein by reference.

Further available to the worker in the art are cDNA libraries of bovine and human liver endothelium (e.g., obtained and developed from Clontech, Palo Alto, Calif.).

Isolation of porcine or human ecto-ATP diphosphohydrolase is carried out by the methods described by Cote et al., id. or Sevingy and co-workers, id., utilizing FSBA labelling and immunodetection. Specific activity of the enzyme is determined as described by LeBel et al., id.

Following the protein purification, the protein sequence of, for example, the bovine species can be determined using standard, commercially available methodology, e.g., an Applied Biosystems Sequerator. Concurrently, polyclonal antibodies are raised against the bovine ATP diphosphohydrolase protein. Monoclonal and/or polyclonal antibodies are raised against the protein by techniques disclosed, for example, by Lin and Guidotti, id. and Cheung et al., id. With monoclonal, and previously described polyclonal, antibodies in hand, together with a knowledge of at least a part of the protein sequence, there are two approaches to obtaining the gene in bovine, porcine or human cells:

(i) Utilizing an expression library, the available antibodies are used to detect the colony including the cDNA encoding for the ATP diphosphohydrolase; and

(ii) Utilizing defined oligomers corresponding to the amino acid sequences that have been obtained, to obtain the correct cDNA elements. See Lin and Guidotti, id. and Cheung et al., id.

The porcine cDNA sequence can be obtained by similar techniques as described above by probing with suitable antibodies or oligomers. Likewise the human ecto-ATP diphosphohydrolase protein can be determined following the procedures defined above, or alternatively by probing human cDNA from endothelial cells or genomic libraries.

Thereafter the entire length of cDNA can be sequenced by known methods (Rosenthal, NE J Med. 332 (9) 589-591).

The obtained native cDNA can also be expressed recombinantly in E. coli.

The above procedures are well-described by Sambrook, Fritsch and Maniatis, Molecular Cloning, A Laboratory Manual, 2d Edition., Cold Spring Harbor Laboratory, Cold Spring Harbor, N.Y., 1989.

The distribution of CD39 protein on B lymphocytes, activated NK cells, and certain T cell and endothelial cell lines (see Plesner, Inter. Rev. Cytology 1995; 158 (141): 141-214; Maliszewski et al., 1994, id.; Kansas et al., J. Immunol. 1991; 146 (7): 2235-44) is consistent with the known distribution of ecto-ADPases. The cell surface glycoprotein CD39 has two potential transmembrane regions, and binding by certain antibodies triggers signal transduction. The reported molecular mass of the native CD39 protein is 70-100 kDa with 6 potential N-glycosylation sites and an observed molecular mass of 54 kDa after enzymatic removal of N-linked sugars (Maliszewski et al., 1994, id.). Additionally, there are several potential targets for oxidative damage as the available deduced sequence data show that the protein is rich in cysteine (n=11), methionine (n=12) and tyrosine (n=27).

CD39 in a similar fashion to other markers is designated as a B cell activation marker (Engel et al., Leukemia & Lymphoma 1994, 1 (61): 61-4). CD39 has been shown to have partial identity with yeast guanosine diphosphatases but no specific function has been yet assigned although a role in the mediation of homotypic B cell adhesion and Ag-specific responses has been described (Maliszewski et al., 1994, id.; Kansas et al., 1991, id.). The antigen has been found expressed on endothelial cells where activation related changes have been mentioned, in association with over 120 other potential markers (Favaloro, Immun. Cell Biol. 1993; 71 (571): 571-581), and has been noted to be expressed on vascular endothelium, particularly in cutaneous vessels (Kansas et al., 1991, id.).

Once the native protein of interest is sequenced, it can be derivatized (i.e. mutated or truncated or otherwise altered by known procedures in the art) for the purpose of increasing resistance to oxidative stress.

Examples of involved physiological oxidants against which oxidation-resistance is desirably maintained are superoxide and hydroxyl radicals and related species such as hydrogen peroxide and hypohalous acid. Oxygen free radical intermediates, such as superoxide and hydroxyl radicals, are produced through normal and pathologic metabolic processes.

Of the amino acids that make up proteins, histidine, methionine, cysteine, tryptophan and arginine are the most likely to be oxidized. For example, oxidation of methionines of a native protein may cause the protein to lose activity. Tyrosine is susceptible to nitric oxide and peroxynitrate, which could also thereby inactivate enzyme function.

Therefore, in such case different amino acids can be substituted for the native methionines, as described by Glaser et al., U.S. Pat. No. 5,256,770, which is incorporated herein by reference.

Methods for rendering amino acids resistant to oxidation are generally known. A preferred method is by removing the affected amino acid or replacing it with one or more different amino acids that will not react with oxidants. For example, the amino acids leucine, alanine and glutamine are preferred replacement amino acids based on size and neutral character.

Methods by which amino acids can be removed or replaced in the sequence of a protein are also well-known to the skilled worker. Genes encoding a peptide with an altered amino acid sequence can be made synthetically, see, e.g., Higuchi, 1990, PCR Protocols, at 177-183, Acad. Press., San Diego. A preferred method comprises site directed in vitro mutagenesis, which involves the use of a synthetic oligodeoxyribonucleotide containing a desired nucleotide substitution, insertion or deletion designed to specifically alter the nucleotide sequence of a single-strand target DNA. This primer, when hybridized to a single-strand template with primer extension, results in a heteroduplex DNA which, when replicated in a transformed cell, encodes a protein sequence with the intended mutation.

A mutant ecto-ATPase analog that retains at least about 60%, and more preferably at least 70%, and even more desirably at least 90%, of normal activity after exposure to oxidants, can be considered to be substantially oxidation-resistant.

This invention also provides for pharmaceutical compositions having anti-platelet aggregatory activity comprising a sterile preparation of a unit dose of a (preferably oxidation-resistant) ecto-ATP diphosphohydrolase soluble analog in a pharmaceutically acceptable carrier.

Administration of such analogs can be by a bolus intravenous injection, by a constant intravenous infusion, or by a combination of both routes.

The invention also contemplates biocompatible materials, such as prosthetic devices, which are coated with an oxidation resistant ecto-ATP diphosphohydrolase analog. See, for example, Ito et al., U.S. Pat. No. 5,126,140, which is incorporated by reference.

The present invention broadly comprises a method of treating the dysfunctional or activation response of a mammalian cell (e.g., an endothelial cell) to an inflammatory or other platelet-mediated activation stimulus, comprising modifying said (endothelial) cell by inserting therein DNA encoding a polypeptide having ATP diphosphohydrolase activity, in operative association with a suitable promoter, and secreting and/or expressing functional ecto-ATPase from said cells at effective levels whereby platelet aggregation at the cell surface is inhibited.

The invention also includes the cells so modified, and tissues or organs comprising said cells.

Cells or cell populations can be treated in accordance with the present invention in vivo or in vitro (ex vivo).

For example, for purposes of in vivo treatments, ecto-ATP diphosphohydrolase vectors can be inserted by direct infection of cells, tissues or organs in situ.

For example, the blood vessels of an organ (e.g., kidney) can be temporarily clamped off from the blood circulation of the patient, and the vessels perfused with a solution comprising a transmissible vector construct containing the subject ecto-ATP diphosphohydrolase gene, for a time sufficient for at least some of the cells of the organ to be genetically modified by insertion therein of the vector construct; and on removal of the clamps, blood flow can be restored to the organ and its normal functioning resumed.

Adenoviral mediated gene transfer into vessels or organs by means of transduction perfusion, as just described, is a means of genetically modifying cells in vivo.

The invention in a further aspect comprises a method for inhibiting platelet aggregation or thrombus formation in a subject in need of such therapy, which comprises inserting into cells of the suject which are subject to platelet-mediated activation or inflammation, DNA encoding a polypeptide having ATP diphosphohydrolase activity, in operative association with a promoter, and expressing said polypeptide at platelet-aggregation (thrombus-suppressing) effective levels.

In another aspect, cell populations can be removed from the subject or a donor animal, genetically modified ex vivo by insertion of vector DNA, and then re-implanted into the subject or transplanted into another recipient.

Thus for example, an organ can be removed from a patient or donor, subjected ex vivo to the perfusion step previously described, and the organ can then be re-grafted into the patient or implanted into a different recipient of the same or different species.

Ex vivo genetically modified endothelial cells may be administered to a patient by intravenous or intra-arterial injection under defined conditions.

In still another embodiment, the invention comprises a method for transplanting donor cells, or tissue or organs comprising said cells, into a mammalian recipient in whom said cells are susceptible to a platelet-mediated activation stimulus, which comprises:

(a) modifying the donor cells, or progenitor cells thereof, by introducing therein DNA encoding a protein having ATP diphosphohydrolase activity; and

(b) transplanting the so-modified donor cells, tissue or organ into the recipient and expressing the polypeptide having ATP diphosphohydrolase activity, whereby recipient platelet aggregation at the surface of the cells is reduced or inhibited.

The donor species may be any suitable species which is the same or different from the recipient species and which is able to provide the appropriate endothelial cells, tissue or organ for transplantation or grafting.

In a preferred embodiment, human ecto-ATP diphosphohydrolase is expressed from cells of a different mammalian species, which cells have been placed or grafted into a human recipient.

The donor may be of a species which is allogeneic or xenogeneic to that of the recipient. The recipient is a mammal, e.g., a primate, and is primarily human. However, other mammals, such as non-human primates, may be suitable recipients.

For human recipients, it is envisaged that human (i.e. allogeneic) as well as pig (i.e. xenogeneic) donors will be suitable, but any other mammalian species (e.g., bovine or non-human primate) may also be suitable as donors.

For example, porcine aortic endothelial cells (PAEC), or the progenitor cells thereof, can be obtained from porcine subjects, genetically modified, and reimplanted into either the autologous donor (until a time suitable to be recruited for transplantation) or transplanted into another mammalian (i.e. human) subject.

The donor cells or tissue may be somatic recombinants or transgenic in the sense that they contain and express DNA encoding ecto-ATP diphosphohydrolase protein of a graft recipient of a different species in whom they are, or will be, implanted. Such cells or tissue may continue to express the desired ecto-ATP diphosphohydrolase indefinitely for the life of the cell.

For example, porcine aortic endothelial cells (PAEC), or the progenitor cells thereof, can be genetically modified to express porcine or human ATP diphosphohydrolase protein at effective levels, for grafting into a human recipient.

Heterologous genes can be inserted into germ cells (e.g., ova) to produce transgenic animals bearing the gene, which is then passed on to offspring. For example, DNA encoding ATP diphosphohydrolase can be inserted into the animal or an ancester of the animal at the single-cell stage or early morula stage. The preferred stage is the single-cell stage although the process may be carried out between the two and eight cell stages.

Methods of preparing transgenic pigs are discussed by Pinckert et al., Xeno, Vol. 2, No. 1, 1994, and the references cited therein.

In another aspect genes can be inserted into somatic/body cells of the donor animal to provide a somatic recombinant animal, from whom the DNA construct is not capable of being passed on to offspring (see, e.g., Miller, A. D. and Rosman, G. T., Biotechniques, 1989, 7, No. 9, 980-990).

Preferably, the inserted DNA sequences are incorporated into the genome of the cell. Alternatively, the inserted sequences may be maintained in the cell extrachromosomally, either stably or for a limited period.

Cells, tissue or organs may be removed from a donor and grafted into a recipient by well-known surgical procedures.

Although any mammalian cell can be targeted for insertion of the ecto-ATP diphosphohydrolase gene, endothelial cells are the preferred cells for manipulation.

Modification of endothelial cells according to the invention can be by any of various means known to the art.

In vivo direct injection of cells or tissue with DNA can be carried out, for example.

Appropriate methods of inserting foreign cells or DNA into animal tissue include microinjection, embryonic stem (ES) cell manipulation, electroporation, cell gun, transfection-k, transduction, retroviral infection, etc.

In another embodiment, the gene is inserted into a particular locus, e.g., the thrombomodulin locus, or locus containing von Willebrand factor. To prepare transgenic animals with such a vector, the construct is introduced into embryonic stem (ES) cells, and the resulting progeny express the construct in their vascular endothelium.

For gene delivery, retroviral vectors, and in particular, replication-defective retroviral vectors lacking one or more of the gag, pol, and env sequences required for retroviral replication, are well-known to the art and may be used to transform endothelial cells. PA 317 or other producer cell lines producing helper-free viral vectors are well-described in the literature.

A representative retroviral construct comprises at least one viral long terminal repeat and promoter sequences upstream of the nucleotide sequence of the therapeutic substance and at least one viral long terminal repeat and polyadenylation signal downstream of the therapeutic sequence.

Vectors derived from adenoviruses, i.e. viruses causing upper respiratory disease and also present in latent infections in primates, are also generally known to the art and are useful in certain circumstances, particlarly in view of their ability to infect nonreplicating somatic cells. The ability of adenoviruses to attach to cells at low ambient temperatures is also an advantage in the transplant setting which can facilitate gene transfer during cold preservation.

Prior to implantation, the treated endothelial cells or tissue may be screened for genetically modified cells containing and expressing the construct. For this purpose, the vector construct can also be provided with a second nucleotide sequence encoding an expression product that confers resistance to a selectable marker substance. Suitable selectable marks for screenng include the neo gene, conferring resistance to neomycin or the neomycin analog, G418.

Alternative means of targeted gene delivery comprise DNA-protein conjugates, liposomes, etc.

The protein encoding region and/or the promoter region of the inserted DNA, may be heterologous, i.e. non-native to the cell. Alternatively, one or both of the protein encoding region and the promoter region may be native to the cell, provided that the promoter is other than the promoter which normally controls ATP diphosphohydrolase expression in said cell.

The protein coding sequence may include sequence coding for an appropriate signal sequence, e.g., a nucleus specific signal sequence.

Means to achieve thrombus-suppressing effective (i.e. “stable”) levels of expression of an ATP hydrolyzing protein such as CD39 under endothelial activating conditions are also available.

Preferably the protein encoding region is under the control of a constitutive or inducible (i.e. a subset of “regulable”) promoters.

An advantage of employing an inducible promoter for transplantation purposes is that the desired high level transcription/expression of the active gene/protein can be delayed for a suitable period of time before grafting. For example, transcription can be obtained on demand in response to a predetermined stimulus, such as, e.g., the presence of tetracycline in the cellular environment.

An example of a tetracycline-inducible promoter which is suitable for use in the invention is disclosed by Furte et al., PNAS 91, 1994, 9302-9306. Alternatively, a regulable promoter system in which transcription is initiated by the withdrawal of tetracycline is described by Gossen and Bujard, PNAS USA 90, 1992, 5547-51.

Preferably, transcription/expression of the ATP diphosphohydrolase gene/protein is induced in response to a predetermined external stimulus, and the stimulus is applied beginning immediately prior to subjecting the cells to an activating stimulus, so that expression is already at effective levels for platelet aggregation-suppressing purposes.

For example, cells of a donor mammal (e., porcine) may be genetically modified according to the invention by insertion of the ATP diphosphohydrolase gene (e.g., porcine or human) under the control of an promoter which is inducible by a drug such as, e.g., tetracycline. The animal, whether a somatic recombinant or a transgenic, may be raised up to the desired level of maturity under tetracycline-free conditions, until such time as said cells, or tissue or organs comprising said cells, are to be surgically removed for transplantation purposes. In such case, prior to surgical removal of the organ, the donor animal may be administered tetracycline in in order to begin inducing high levels of transcription/expression of the ATP hydrolyzing gene/protein. The organ can then be transplanted into a recipient (e.g., human), and tetracycline may continue to be administered to the recipient for a sufficient time to maintain the ATP diphosphohydrolase protein at the desired levels in the transplanted cells to inhibit platelet aggregation in the recipient.

Alternatively, the organ after being surgically removed from the donor, can be maintained ex vivo in a tetracycline-containing medium until such time as grafting into a recipient is appropriate.

In another embodiment, transcription may be provided to occur as a result of withholding tetracycline from the cellular environment. Thus, cells of a donor animals may be genetically modified according to the invention by insertion of a gene encoding an ATP diphosphohydrolase protein under the control of a promoter which is blocked by tetracycline, and which is induced in the absence of tetracycline. In such case, the animal may be raised up to the desired level of maturity while being administered tetracycline, until such time as the cells, tissues of organs of said animals are to be harvested. Prior to surgical removal, the donor animal may be deprived tetracycline in order to begin inducing expression of ATP diphosphohydrolase protein, and the patient in whom said cells, tissue or organs are transplanted may thereafter also be maintained tetracycline-free for a sufficient time to maintain appropriate ATP diphosphohydrolase levels of expression.

In addition to using a constitutive or inducible promoter facilitating higl level expression, multiple copies of DNA encoding ATP diphosphohydrolase may be placed in operative association with such a promoter to further increase gene transcription and protein expression.

It will be appreciated that the modified cells and donor tissues and organs defined above have a supplementary function in the prevention of transplant rejection in xenotransplantation since the primary rejection is hyperacute rejection. Therefore, the genetic material of the cells of the donor organ is typically also altered such that activation of the complement pathway in the recipient is prevented. This may be done by providing transgenic animals that express the complement inhibitory factors of the recipient species. The endothelial cells of a donor organ obtained from such an animal can be modified by gene therapy techniques to provide the endothelial cells defined above. Alternatively a vector containing DNA encoding a protein having ATP diphosphohydrolase activity can be introduced into the transgenic animal at the single cell stage or early morula stage. In this way, the resulting transgenic animal will express the complement inhibitory factors and will have endothelial cells as defined above. Thus in a further aspect the invention also provides endothelial cells, tissue, donor organs and non-human transgenic or somatic recombinant animals as defined above which express one or more human complement inhibitory factors.

Although any mammalian cell can be targeted for insertion of the ATP diphosphohydrolase gene, such as monocytes, NK cells, lymphocytes, or islet cells, the preferred cells for manipulation are endothelial cells.

In an alternative embodiment of the invention, the polypeptide having ATP diphosphohydrolase activity, in a pharmaceutically acceptable carrier, may be applied directly to cells, tissues or organs in vivo.

Thus the invention also comprises a method of inhibiting platelet aggregation in a warm-blooded mammal comprising administering to said mammal an effective amount for inhibiting platelet aggregation of a polypeptide having ATP diphosphohydrolase activity, or pharmaceutically acceptable salt thereof, in a pharmaceutically acceptable carrier.

In particular, the invention comprises a method of inhibiting platelet aggregation in a warm-blooded mammal comprising administering to said mammal an effective amount for inhibiting platelet aggregation of CD39, or a pharmaceutically acceptable salt thereof, in a pharmaceutically acceptable carrier.

The invention additionally comprises a pharmaceutical composition having anti-platelet aggregatory activity comprising a unit dose of a polypeptide having ATP diphosphohydrolase activity (e.g., CD39), or pharmaceutically acceptable salt thereof, in a pharmaceutically acceptable carrier.

A polypeptide according to the invention or a hydrohalic addition product thereof is typically adminsitered as a pharmaceutical composition in the form of a solution or suspension. However, as is well known, peptides can also be formulated for therapeutic administration as tablets, pills, capsules, sustained release formulations or powders. The preparation of therapeutic compositions which comprise polypeptides as active ingredients is well understood in the art. Typically, such compositions are prepared as injectables, either as liquid solutions or as suspensions.

A therapeutic composition useful in the practice of the present invention can contain a polypeptide having ATP diphosphohydrolase activity formulated into a therapeutic composition as a neutralized pharmaceutically accepable salt form. Pharmaceutically acceptable salts include acid addition salts (formed with the free amino groups of the polypeptide), and which are formed with inorganic acids such as, for example, hydrochloric or phorphoric acid, or such organic acids as acetic, oxalic, tartaric, mandelic, and the like. Salts formed with the free carboxyl groups can also be derived from inorganic bases, such as, for example, sodium, potassium, ammonium, calcium or ferric hydroxides, or such organic bases as isopropylamine, trimethylamine, 2-ethylamino ethanol, hisidine, procaine, and the like.

The therapeutic peptide-containing composition is conventionally administered intravenously, as by injection of a unit dose, for example.

The term “unit dose” when used in reference to a therapeutic composition used in the present invention refers to physically discrete units suitable as unitary dosages for humans, each unit containing a predetermined quantity of active material calculated to produce the desired therapeutic effect in association with the required excipient.

The composition is administered in a manner compatible with the dosage formulation, and in a therapeutically effective amount. The quantity to be administered depends on the subject to be treated, capacity of the subject's blood hemostatic system to utilize the active ingredient, and the degree of platelet aggregation inhibition desired. The precise amount of active ingredient required to be administered depends on the judgment of the practitioner and is peculiar to each individual. However, suitable dosage ranges are of the order of one to hundreds of nanomoles of polypeptide per kilogram body weight per minute, and depend on the route of administration.

Also contemplated to be within the scope of the invention is a vascular prothesis having applied thereto a polypeptide having ATP diphosphohydrolase activity (e.g., CD39). Commercially available materials suitable for fabircating such a prosthesis include a polyester such as Dacron (C. R. Bard) or a polyfluorocarbon such as Teflon (Gore-Tex).

The present invention may be applied in the therapeutic treatment of a wide variety of disease states in mammals where there is an increase in propensity for platelet aggregation (e.g., atherosclerotic and thrombotic conditions, such as ischemic heart disease, atherosclerosis, multiple sclerosis, intrcranial tumors, thromboembolism and hyperlipemia, thrombophlebitis, phlebothrombosis, cerebral thrombosis, coronary thrombosis and retinal thrombosis), as well as following parturition or surgical operations such as coronary artery bypass surgery, angioplasty, or prosthetic heart valve implantation.

The following examples are intended to be illustrative only and not limitative of the invention.

### EXAMPLE 1(A)

Xenogeneic quiescent porcine aoric endothelial cells (PAEC) in the absence of plasma XNA and C exert an inhibitory effect on human platelet activation responses to standard platelet agonists.

The factor inhibitory to human platelet activation in in vitro systems is cell-associated and not found in cell culture supernatants. This cell associated factor completely blocks human platelet responses to ADP (2-10 μM), collagen (2-10 μg/ml) and low concentrations of thrombin (<1 U/ml) in the presence of PAEC in monolayer, on bead cultures or cell suspensions.

The importance of prostacycline metabolites, thrombomodulin (by thrombin neutralization) and NO have been evaluated by several methodologies and shown not to be crucial for this inhibition of platelet activation processed by PAEC.

Because of the demonstrable non-inhibitable effects of ADP-β-S (a non-hydrolyzable analogue of ADP which is thus not degraded by the ecto-ADPases) on human platelet responses in association with PAEC in the experimental systems examined, the inhibitory endothelial cell associated factor is identified as an ecto-ATP diphosphohydrolase (apyrase).

### EXAMPLE 1(B)

The inhibitor phenotype of PAEC is lost following PAEC activation.

Activation of PAEC by standardized human recombinant TNF in vitro results in rapid loss, by 30 to 60 minutes, of the EC antiaggregatory phenotype with the development of a permissive environment for platelet activation.

### EXAMPLE 1(c)

Modulation of Ecto-ATP Diphosphohydrolases on Porcine Aortic Endothelial Cells by TNFa.

The endothelial cell ecto-ATP diphosphohydrolase is significantly modulated by EC activation responses.

Kinetics of ecto-ATP diphosphohydrolase: As determined by catabolism of 14^{C} ADP, PAEC ecto-ATP diphosphohydrolase Vmax is of the order of 50-55 nmol ADP converted per 1×10 6 cells/min (Km approximately 200 uM). These figures are in concordance with those stated for human umbilical vein EC and previously for porcine EC as determined by other methodology (Marcus et al., J Clin Invest 1991; 88: 1690; Gordon et al., J Biol Chem 1986; 261: 15496-15507).

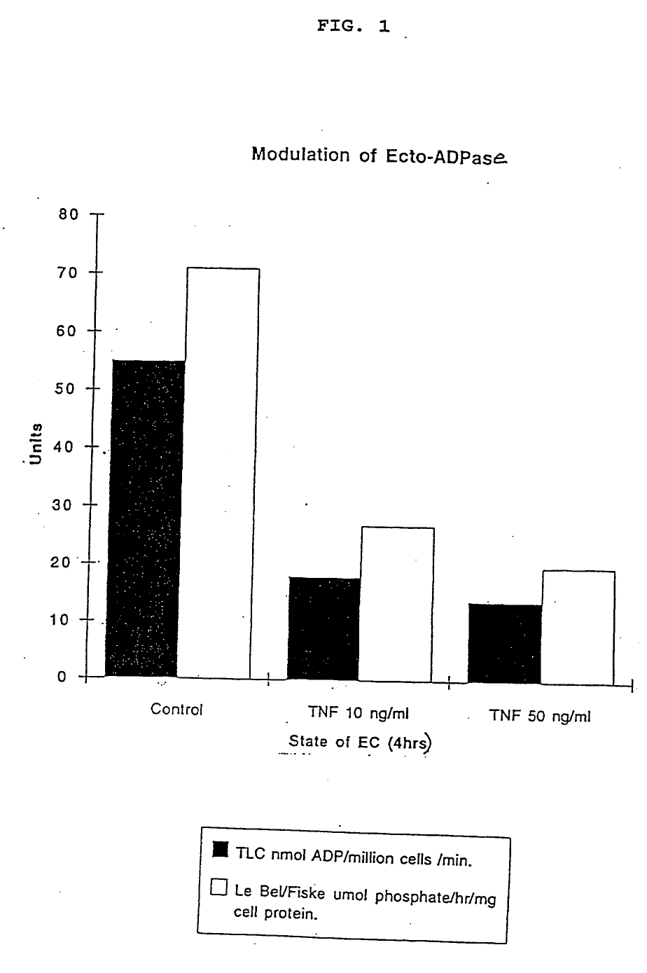

Endothelial cells when activated by TNFα at 10 and 50 ng/ml lose ecto-ADPase activity after 60 minutes incubation. FIG. 1 shows levels of enzyme activity at 4 hours as determined by biochemical methodology (LeBel et al. J. Biol. Chem. (1980) 255, 1227-1233) as well as TLC determination of cellular degradation of C¹⁴-ADP to AMP (Marcus et al., J. Clin. Investig. (1991) 88: 1690-1696). Once EC are activated, there is loss of this inhibitory potential, and therefore platelet activation can occur. This inhibitory activity is chiefly related to ecto-ATP diphosphohydrolase expressed on PAEC.

### EXAMPLE 1(d)

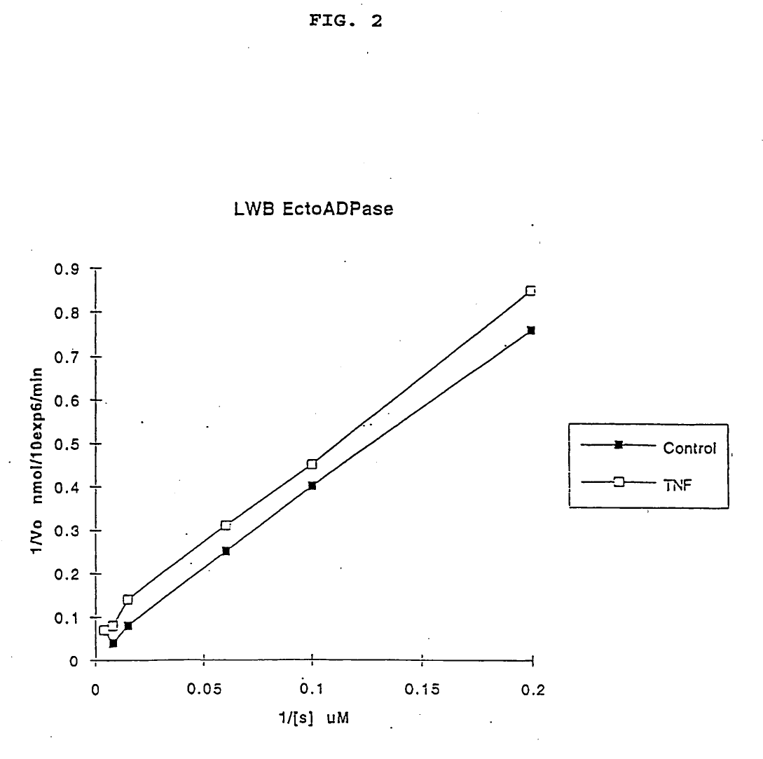

PAEC ecto-ATP diphosphohydrolase kinetics post-activation of intact cells were also determined by TLC: Vmax 15 nmolADP/1×106 cells/min (Km 70 μM). Reciprocal plots suggest an uncompetitive inhibition process. This novel observation is in keeping with either an inhibitor binding to the enzyme-substrate complex (but not the free enzyme itself) or a process of inhibition which disturbs the enzyme catalytic function independent of substrate binding. (FIG. 2).

### EXAMPLE 2(A)

Oxidative stress inhibits porcine endothelial cell ecto-ATP diphosphohydrolase.

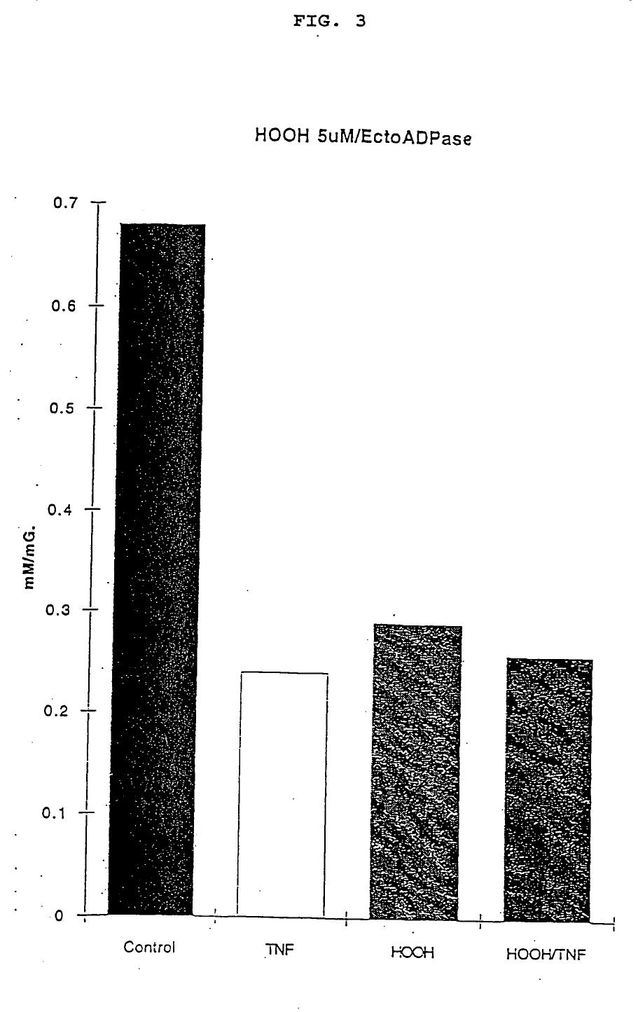

Incubation of PAEC with HOOH at concentrations of 5 μM and 10 μM which are potentially produced by activated endothelial cells, in the absence of catalase activity, has a significant effect on the activity of the ecto-ATP diphosphohydrolase comparable and non-additive to that observed following cell activation with cytokines. FIG. 3 depicts loss of enzyme activity after treatment with 5 uM HOOH after 4 hours incubation.

The generation of HOOH by PAEC following activation with cytokines such as TNF in vitro was determined to be of the order of about 0.015 nmoles/min/10⁶ cells.

Ecto-ATP diphosphohydrolases could thus be sensitive to oxidation processes which are promoted by cytokine activation of PAEC. Endogenous xanthine oxidase and other, e.g., NADPH oxidase, enzyme systems in PAEC elaborate significant levels of reactive oxygen intermediates following cellular activation and these could have profound effects on membrane associated ectoenzymes.

### EXAMPLE 2(B)

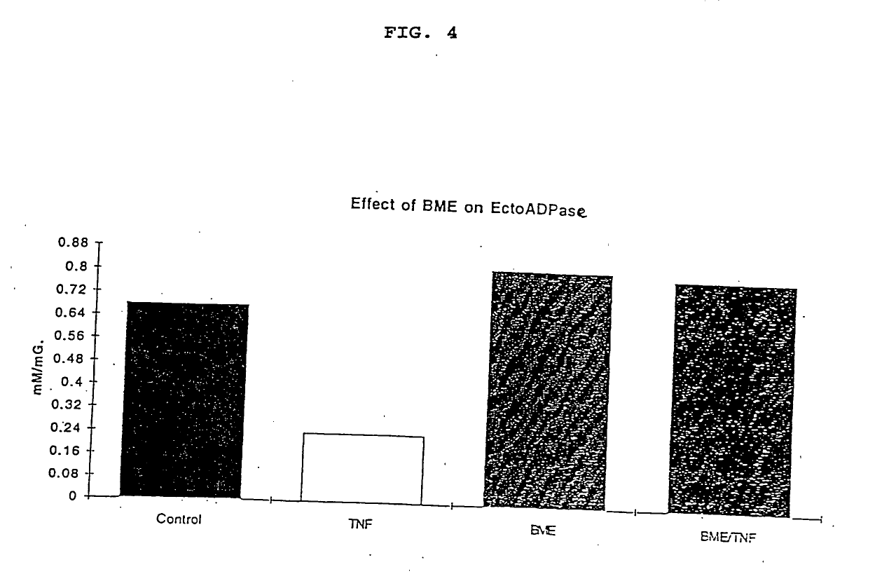

In a reciporocal fashion to agents which induce oxidative stress, beta mercaptoethanol a potent antioxidant and reducing agent in micromolar concentrations, protects the enzyme activity. This also holds for situations under which endothelial cells are activated by cytokines (FIG. 4).

### EXAMPLE 2(c)

A loss of ecto-ATP diphosphohydrolase activity on PAEC is demonstrated as a result of TNFα activation and following incubation with and perturbation of endothelial cells by HOOH (peroxide 5 μM) and by Xanthine Oxidase/Xanthine (XO/X at combinations of 200 μM xanthine and typically 100 mU/ml of xanthine oxidase which is phosphate free) in vitro. XO/X cause oxidative damage to cells and their membrane proteins and lipids by both peroxide and superoxide radicals. In the presence of iron, toxic hydroxyl radicals are formed. Note the late decrease in enzyme activity following exposure to oxygen radicals (FIG., 5).

### EXAMPLE 3

Antioxidant strategies with SOD/catalase supplementation in the systems tested likewise are shown to be protective in preserving endothelial cell ecto-ATP diphosphohydrolase activity following activation processes. Superoxide dismutase (Cu—Zn form from Bovine RBC) removes oxygen radicals, and was used at a concentration of 330 u/ml. Catalase degrades HOOH, and a preparation from bovine liver was used at a final concentration of 1,000 u/ml.

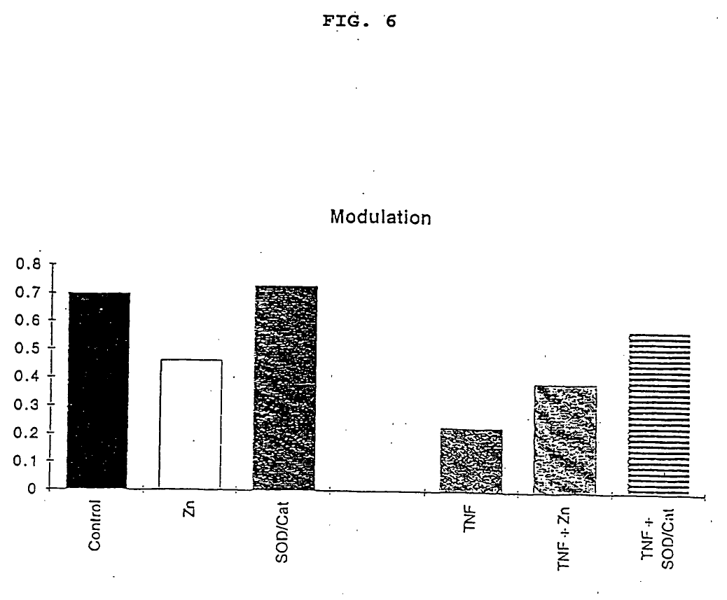

Zinc has protean effects on cell membranes but can also serve as a potent antioxidant as potentially demonstrated here at concentrations previously documented to maintain porcine endothelial integrity following cytokine perturbation in vitro. Supplementation in these systems likewise appear to be protective in preserving endothelial cell ecto-ATP diphosphohydrolase activity (FIG. 6).

### EXAMPLE 4

Direct oxidation of the endothelial cell ecto-ATP diphosphohydrolase is responsible for the modulation of endothelial cell-platelet interactions in the setting of cellular activation.

Experiments similar to those described above on the purified protein are performed to evaluate further the direct loss of activity following oxidation with or without further proteolytic modification, Rivett, Curr Top Cell Regul 1986; 28: 291).

### EXAMPLE 5

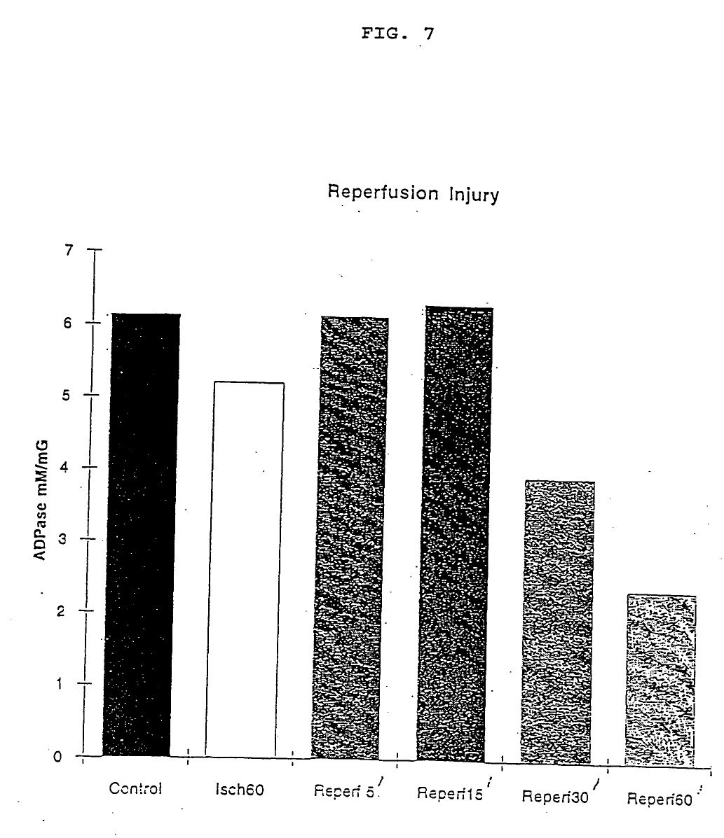

FIG. 7 demonstrates loss of activity after 60 minutes warm ischaemic time and then in addition 5, 15, 30 and 60 minutes warm reperfusion in vivo. Note the loss in activity after 30 minutes reperfusion in vivo. Initial increases in ATP diphosphohydrolase activity could represent associated leucocyte adherence to injured endothelium in vivo.

### EXAMPLE 6

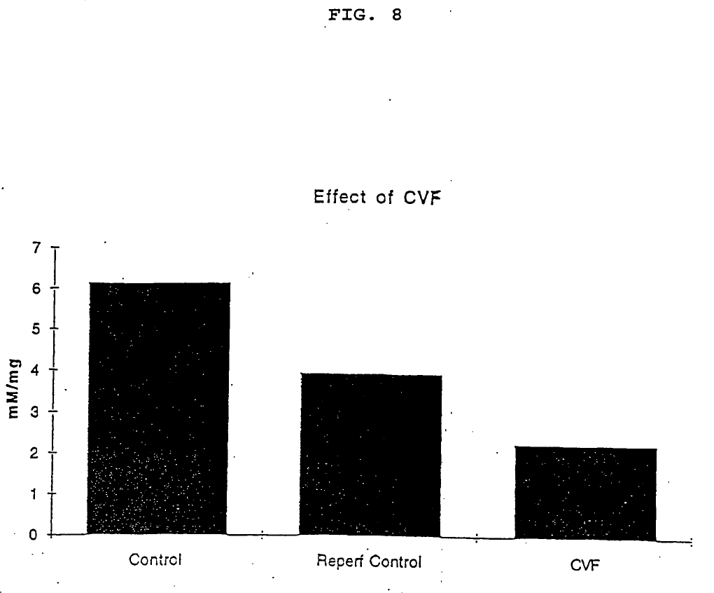

FIG. 8 demonstrates that pretreatment of rats with cobra venom factor to deplete animals of complement also results in systemic complement activation injury and as a consequence potentiates the loss of ATP diphosphohydrolase activity when glomeruli are rendered ischaemic and then reperfused for 30 minutes.

### EXAMPLE 7

Northern Analysis of CD39 in HUVEC following cytokine activation.

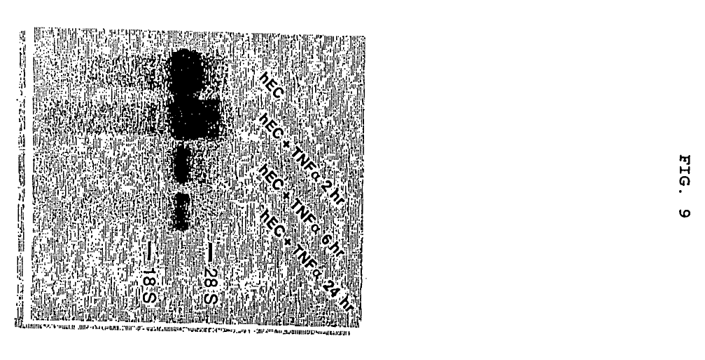

Human umbilical vein endothelial cells (HUVEC) were incubated with TNFα (final concentration 10 ng/ml) for 2, 6 and 24 hours. Cells were washed twice with a phosphate buffer, RNA was purified and analysed by Northern blot. Ten μg of total RNA per well was applied on the TAE-Agarose gel. Electrophoresis was run at 40 mA for 2 hours. RNA was transferred to a charge-modified nylon membrane and UV-cross linked. CD39 cDNA fragment cleaved from the plasmid DNA (pCDNA3-CD39) was labeled with [α³²P]-dCTP to a specific activity of 2×10⁹ cpm/μg DNA, by the random hexamer labeling method. Prehybridization, hybridization, washes, and stripping of the membrane were carried out with the rapid hybridization protocol from Stratagene. Final washes were at 60° C. in 0.1-×sodium saline citrate (SSC)/0.1% SDS. The blot was exposed to Kodak XAR-2 film with an intensifying screen at −80° C. for 1 day. Our results as depicted in FIG. 9 show markedly decreased levels of CD39/ecto-ADPase mRNA following TNFα stimulation of EC at 6 hours and beyond to 24 hours.

What is claimed is:

1. A method of genetically modifying a mammalian cell to render it capable of inhibiting platelet aggregation, which comprises: inserting in said cell, or a progenitor thereof, DNA encoding a polypeptide having activity of an ATP diphosphohydrolase, and expressing said polypeptide from said cell at platelet aggregation-suppressing effective levels.

2. The method of claim 1 wherein the polypeptide having activity of an ATP diphosphohydrolase comprises CD39.

3. The method of claim 2 wherein the polypeptide comprises human CD39.

4. The method of claim 3 wherein the polypeptide is substantially oxidation-resistant.

5. A human endothelial cell modified according to the method of claim 1.

6. A porcine endothelial cell modified according to the method of claim 1.

7. A human endothelial cell modified according to the method of claim 3.

8. A porcine endothelial cell modified according to the method of claim 3.

9. A method of controlling platelet aggregation and thereby preventing or alleviating a thrombotic condition in a mammalian subject which comprises: inserting into cells of the subject susceptible to platelet-mediated activation, DNA encoding a polypeptide having ATP-diphosphohydrolase activity, and expressing from said cells said polypeptide at platelet-aggregation suppressing effective levels.

10. The method of claim 9 in which the DNA is inserted into endothelial cells.

11. The method of claim 10 in which the polypeptide having activity of an ATP diphosphohydrolase comprises human CD39.

12. The method of claim 11 wherein the subject is human.

13. The method of claim 11 in which the polypeptide is substantially oxidation resistant.

14. A method of transplanting donor endothelial cells, or graftable tissue or an organ comprising said cells, to a mammalian recipient in whose blood such cells, tissues or organs are susceptible to an activation stimulus, which comprises:
(a) genetically modifying the donor cells, or progenitor cells thereof, by inserting therein DNA encoding a polypeptide having activity of an ATP diphosphohydrolase; and
(b) transplanting the so-modified donor cells, tissue or organ into the recipient, and expressing from said cells the polypeptide having ATP diphosphohydrolase activity at platelet-agregation suppressing effective levels.

15. The method of claim 14 in which the polypeptide having activity of an ATP diphosphohydrolase comprises human CD39.

16. The method of claim 15 in which the recipient is human.

17. The method of claim 14 in which the polypeptide is substantially oxidation resistant.

18. The method of claim 16 in which the donor is xenogenic as to the recipient.

19. The method of claim 16 in which the donor cells, tissue or organs are porcine.

20. A method of inhibiting platelet aggregation in a mammal comprising administering to said mammal an effective amount for inhibiting platelet aggregation of a polypeptide having ATP diphosphohydrolase activity, or pharmaceutically acceptable salt thereof, in a pharmaceutically acceptable carrier.

21. The method of claim 20 wherein the polypeptide having ATP diphosphohydrolase activity comprises human CD39.

22. The method of claim 21 wherein the mammal is a human.

23. A pharmaceutical composition having anti-platelet aggregatory activity comprising a unit dose of a polypeptide having ATP diphosphohydrolase activity, or pharmaceutically acceptable salt thereof, in a pharmaceutically acceptable carrier.

24. The pharmaceutical composition of claim 23 wherein the polypeptide having ATP diphosphohydrolase activity is human CD39.

25. A non-human transgenic mammal comprising DNA encoding a polypeptide having activity of an ATP diphosphohydrolase of a different species.

26. The non-human transgenic mammal of claim 25 in which the polypeptide comprises human CD39.

27. The non-human transgenic mammal of claim 26 which is porcine.

28. A non-human transgenic mammal of claim 27 in the polypeptide comprises an oxidation resistant analog of human CD39.

29. Mammalian endothelial cells, tissue or organs capable of expressing DNA encoding a polypeptide having ATP diphosphohydrolase activity at platelet-suppressing effective levels under cellular activating conditions.

30. Graftable endothelial cells, tissue or organs of a donor mammalian species, the cells, tissue or organs being modified to express ATP diphosphohydrolase of a xenogeneic graft recipient species under cellular activation conditions.

31. A prosthetic intravascular device comprised of a synthetic biocompatible material having applied thereto recombinant ATP-diphosphohydrolase or an oxidation-resistant analog thereof.
